(19) United States
(12) Patent Application Publication
(10) Pub. No.: US 2025/0170118 A1
(43) Pub. Date: May 29, 2025

(54) PHARMACEUTICAL COMPOSITION FOR PREVENTING OR TREATING WET AGE-RELATED MACULAR DEGENERATION

(71) Applicant: UIF (University Industry Foundation), Yonsei University, Seoul (KR)

(72) Inventors: Junwon Lee, Seoul (KR); Suk Ho Byeon, Seoul (KR); Gang Cui, Seoul (KR)

(21) Appl. No.: 18/958,703

(22) Filed: Nov. 25, 2024

(30) Foreign Application Priority Data
Nov. 27, 2023 (KR) 10-2023-0166404

(51) Int. Cl. A61K 31/455; A61K 9/00; A61K 31/7064; A61P 27/02

(52) U.S. Cl. CPC A61K 31/455; A61K 9/0014; A61K 31/7064; A61P 27/02

(57) ABSTRACT

This disclosure relates to pharmaceutical compositions for the prevention or treatment wet age-related macular degeneration. In one aspect, the disclosure provides pharmaceutical composition for preventing or treating wet age-related macular degeneration comprising nicotinamide, or derivatives thereof, as an active ingredient. Methods of treating or preventing wet age-related macular degeneration through administration of nicotinamide and derivatives thereof is also described.

### CROSS-REFERENCE TO RELATED APPLICATIONS

This application claims the benefit of Korean Patent Application no. 10-2023-0166404, filed Nov. 27, 2023, which is hereby incorporated herein by reference in its entirety.

### TECHNICAL FIELD

The present invention relates to pharmaceutical compositions for the prevention or treatment wet age-related macular degeneration.

### BACKGROUND ART

The retina is composed of multiple layers, and the most critical layer associated with the pathology of age-related macular degeneration (AMD) is the retinal pigment epithelium (RPE). The RPE interacts with photoreceptors on one side and is adjacent to Bruch's membrane and the choroid on the other side. The RPE plays essential roles for photoreceptors and choroidal capillaries through functions such as light absorption, heat exchange, vitamin A metabolism, phagocytosis of photoreceptor outer segments, and the secretion of vascular endothelial growth factor (VEGF), which maintains the choroidal capillary endothelium.

The accumulation of environmental stress due to aging leads to oxidative stress and inflammation, resulting in dysfunction and death of the RPE. This degeneration of retinal pigment epithelial cells is central to the pathology of AMD, and during fundus examination, yellow drusen are typically observed in the retina. Drusen are accumulations of extracellular material between the RPE and the inner collagen of Bruch's membrane. AMD is classified into early, intermediate, and late stages based on the number and size of drusen and pigmentary changes. In the late stage, it can be divided into dry and wet forms.

Dry (non-exudative) macular degeneration, which accounts for about 90% of all cases, is characterized by large drusen invading extensive areas, resulting in pigmentary changes, loss of RPE, and subsequent loss of photoreceptors. As patchy atrophy of the RPE progresses, geographic atrophy occurs, leading to cell loss and vision decline. On the other hand, wet (exudative) macular degeneration, which comprises about 10% of all AMD cases, primarily develops from dry AMD and is characterized by RPE detachment and neovascularization.

These new blood vessels cause exudates and bleeding in the macula, which is particularly important to our vision, leading to the formation of fibrous scars and resulting in bleeding and swelling. This affects central vision and can lead to blindness. The progression of wet macular degeneration is very rapid, with vision deteriorating quickly within weeks. Therefore, early diagnosis and treatment are extremely important.

Thus, the present invention is designed to solve the aforementioned problems and relates to a pharmaceutical composition for the prevention or treatment of wet (exudative) age-related macular degeneration. The pharmaceutical composition of the present invention has a remarkably significant effect in inhibiting neovascularization and fibrous scarring in wet age-related macular degeneration, and it is expected to be widely used in the medical field.

### SUMMARY

The present invention relates to a pharmaceutical composition for preventing or treating wet age-related macular degeneration (AMD) comprising nicotinamide, or derivatives thereof, as an active ingredient. AMD is classified into early, intermediate, and late stages based on the number and size of drusen and pigmentary changes. In the late stage, it can be divided into dry and wet forms. Among these, wet (exudative) macular degeneration causes exudates, bleeding, and other issues in the macula, which is particularly important in the retina of our eyes, leading to the formation of fibrous scars that can result in blindness. Additionally, the progression of wet macular degeneration is very rapid, with vision deteriorating sharply within a matter of weeks. Therefore, early diagnosis and treatment are crucial.

The pharmaceutical composition of the present invention has a remarkably significant effect in inhibiting neovascularization and fibrous scarring in wet age-related macular degeneration, and it is expected to be widely used in the medical field.

### DISCLOSURE

An object of the present invention is to provide a method for preventing, ameliorating, or treating of wet age-related macular degeneration.

To this end, one aspect of the present invention provides a pharmaceutical composition for the prevention or treatment of wet age-related macular degeneration comprising nicotinamide, or a derivative thereof, as an active ingredient.

In another aspect of the present invention, there is provided a food composition for the prevention or amelioration of wet age-related macular degeneration comprising nicotinamide, or a derivative thereof, as an active ingredient.

In another aspect of the present invention, there is provided a topical formulation for the prevention or treatment of wet age-related macular degeneration comprising nicotinamide, or a derivative thereof, as an active ingredient.

However, objects of the present invention are not limited to the objects mentioned above, and other objects not mentioned herein may be clearly understood by those of ordinary skill in the art from the following description.

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present invention pertains.

In one embodiment of the present invention, “Age-related macular degeneration (AMD)” refers to a disease that occurs with various changes in the macula of the retina as a person ages. The incidence increases with age, and it is known to be the leading cause of adult blindness in developed countries, with approximately 200 million patients worldwide. Therefore, it is expected that the incidence will increase in South Korea as the elderly population grows in the future. The macula refers to the central part of the retina, which is a nervous tissue, densely populated with photoreceptor cells that are responsible for central vision. The retina is composed of multiple layers, and the most critical layer related to the pathology of AMD is the retinal pigment epithelium (RPE). The RPE interacts with photoreceptor cells on one side and is located adjacent to Bruch's membrane and the choroid on the other side. The RPE performs essential functions for photoreceptors and choroidal capillary endothelium through light absorption, heat exchange, vitamin A metabolism, phagocytosis of photoreceptor outer segments, and secretion of vascular endothelial growth factor (VEGF) to maintain the choroidal capillary endothelium.

The accumulation of environmental stress due to aging induces oxidative stress and inflammation, leading to dysfunction and death of the RPE. This degeneration of retinal epithelial cells is the central pathology of AMD, and yellow drusen are typically observed in the retina during fundus examination. Drusen is the accumulation of extracellular material between the RPE and the inner collagen of Bruch's membrane. AMD is classified into early, intermediate, and late stages based on the number and size of drusen and pigmentary abnormalities, and in the late stage, it can be divided into dry and wet types. Dry (non-exudative) macular degeneration, which accounts for about 90% of all cases, is characterized by large drusen invading extensive areas, specific pigmentary abnormalities, loss of the retinal pigment epithelium, and resulting photoreceptor loss. If the atrophy of the RPE becomes patchy, geographic atrophy can occur, leading to vision loss due to cell loss. On the other hand, wet (exudative) macular degeneration, which accounts for about 10% of total macular degeneration, primarily develops from dry AMD and is characterized by detachment of the RPE and neovascularization. These neovascularization cause exudation and hemorrhage, particularly in the macula, which is crucial for our vision, forming fibrous scars and leading to bleeding and edema. This affects central vision and can lead to blindness. Exudative macular degeneration progresses very rapidly, with vision deteriorating significantly within weeks. Therefore, early diagnosis and treatment are very important.

In one embodiment of the present invention, “nicotinamide” refers to a type of vitamin B complex that is widely distributed in plants alongside nicotinic acid and exists as a component of the redox coenzymes NAD+ or NADP+ in animal organisms. In a narrower sense, it is also referred to as niacin.

Nicotinamide, also known as niacinamide, is found in food and is a form of vitamin B3 used as dietary supplements and medications. As a supplement, it is used orally to prevent and treat pellagra, a disease caused by niacin deficiency.

The aforementioned nicotinamide is represented by the following compound of formula 1.

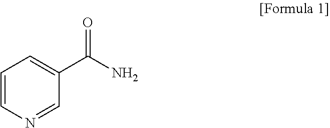

In one embodiment of the present invention, “derivatives of nicotinamide” refer to compounds that are formed by modifying a portion of the molecular structure of nicotinamide. While not limited thereto, it may include nicotinamide mononucleotide, represented by the following compound of formula 2, or nicotinamide riboside, represented by the following compound of formula 3.

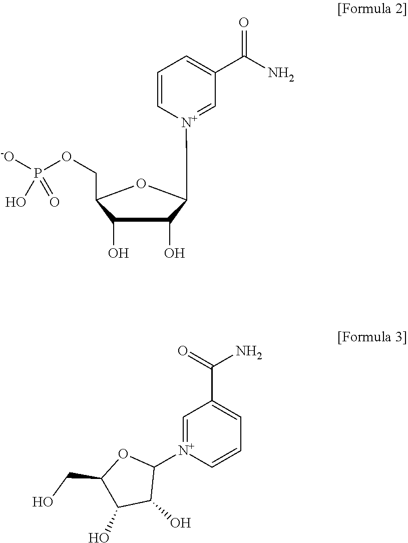

In one embodiment of the present invention, “pharmaceutical compositions” refer to compositions administered for a specific purpose. For the purpose of the present invention, the pharmaceutical compositions aim to prevent or treat wet age-related macular degeneration and may include compounds involved in this, along with pharmaceutically acceptable carriers, excipients, or diluents. Furthermore, the pharmaceutical composition according to the present invention contains 0.1 to 50 weights % of the active ingredient of the present invention relative to the total weight of the composition. Carriers, excipients, and diluents that may be included in the composition of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil, but are not limited thereto.

Specifically, the pharmaceutical composition for the prevention and treatment of wet age-related macular degeneration of the present invention preferably includes any one compound selected from the compound formula 1 to formula 3, or compounds selected from pharmaceutically acceptable salts, optical isomers, hydrates, and solvates thereof.

The pharmaceutically acceptable salts should have low toxicity to the human body and should not adversely affect the biological activity and physicochemical properties of the parent compound. Pharmaceutically acceptable salts can include acid-base salts of pharmaceutically acceptable acids and bases, but are not limited thereto.

A preferred salt form of the compound according to the present invention can include salts with inorganic or organic acids. In this case, inorganic acids such as hydrochloric acid, sulfuric acid, nitric acid, phosphoric acid, perchloric acid, and bromic acid may be used. Additionally, organic acids such as acetic acid, methanesulfonic acid, ethane sulfonic acid, p-toluene sulfonic acid, fumaric acid, maleic acid, malonic acid, phthalic acid, succinic acid, lactic acid, citric acid, gluconic acid, tartaric acid, salicylic acid, malic acid, oxalic acid, benzoic acid, embolic acid, aspartic acid, and glutamic acid may be used. Organic bases that can be used for the preparation of organic base addition salts include tris(hydroxymethyl)methylamine and dicyclohexylamine. Amino acids that can be used for the preparation of amino acid addition salts include natural amino acids such as alanine and glycine. It will be apparent to those skilled in the art that other acids or bases may be used in addition to the inorganic acids, organic acids, organic bases, and amino acids mentioned above. Furthermore, the salts can be prepared by conventional methods.

On the other hand, the compounds according to the present invention may have asymmetric carbon centers, and thus may exist as R or S isomers or racemic compounds, and all of these optical isomers and mixtures are included within the scope of the present invention. In addition, hydrates or solvates of the compounds represented by compound Formula 1 to Formula 3 may also be included within the scope of the present invention.

Meanwhile, in this invention, ‘prevention’ includes any action that blocks, suppresses, or delays symptoms caused by wet age-related macular degeneration using the pharmaceutical composition of this invention, without limitation. Furthermore, in this invention, ‘treatment’ includes any action that improves or benefits symptoms caused by wet age-related macular degeneration using the pharmaceutical composition of this invention, without limitation. The pharmaceutical composition in this invention may be characterized as being in the form of capsules, tablets, granules, injectable forms, ointments, powders, gels, or beverages, and preferably targets animals, more preferably mammals, and most preferably humans.

In one embodiment of the present invention, “administration” refers to introducing the composition of the present invention into a patient by any suitable method. The composition of the present invention may be administered by any general route, as long as it can reach a target tissue. Specifically, it can be administered in the form of oral administration, intraperitoneal administration, intravenous administration, intramuscular administration, subcutaneous administration, intradermal administration, intranasal administration, intrapulmonary administration, intrarectal administration, intracavitary administration, intraperitoneal administration, intrathecal administration, or intraocular administration, and the intraocular administration can be more specifically administered in the form of intravitreal injection, retrobulbar injection, subconjunctival injection, sub-tenon injection, or intraocular eye drops, but is not limited thereto. In the present invention, the effective amount may be adjusted depending on various factors, including the type of disease, the severity of the disease, the types and contents of the active ingredient and other ingredients contained in the composition, the type of formulation, the patient's age, body weight, general health status, sex and diet, the time of administration, the route of administration, the secretion rate of the composition, the duration of treatment, and concurrently used drugs. For adults, the pharmaceutical composition for treatment may be administered into the body in an amount of 50 ml to 500 ml at a time, the compound may be administered at a dose of 0.1 ng/kg to 10 mg/kg, and a monoclonal antibody may be administered at a dose of 0.1 ng/kg to 10 mg/kg. Administration may be performed once to 12 times a day, and when administration is performed 12 times a day, administration may be performed once every 2 hours. In addition, the pharmaceutical composition of the present invention may be administered alone or in combination with other therapies known in the art, such as chemotherapy, radiotherapy, and surgery, for the treatment of the cancer stem cells of interest. The pharmaceutical composition of the present invention may also be administered in combination with other treatments designed to enhance immune responses, e.g., by co-administration with adjuvants or cytokines (or nucleic acids encoding cytokines), as is well known in the art. Other standard delivery methods, e.g., biolistic transfer or ex vivo treatment, may also be used. In ex vivo treatment, antigen presenting cells (APCs), dendritic cells, peripheral blood mononuclear cells, or bone marrow cells may be obtained from a patient or an appropriate donor and activated ex vivo with the pharmaceutical composition of the present invention, and then administered to the patient.

In one embodiment of the present invention, the “food composition” refers to a composition that is used in various ways to prevent or ameliorate the indications targeted by the present invention, and for the purpose of the present invention, the food composition of the present invention is for preventing or improving wet age-related macular degeneration. The food composition containing the composition of the present invention as an active ingredient may be prepared as various foods, for example, beverages, gums, teas, vitamin complexes, powders, granules, tablets, capsules, confectionery, cakes, bread, and the like. Since the food composition of the present invention is an improved food composition containing existing food ingredients with little toxicity and side effects, it may be used with confidence even when it is administered for a long period of time for preventive purposes. When the composition of the present invention is contained in the food composition, it may be added in an amount of 0.1 to 100 wt. % based on the total weight. When the food composition is prepared as a beverage, there is no particular limitation, except that the beverage contains the food composition at the indicated percentage. The beverage may additionally contain various flavorings or natural carbohydrates, like conventional beverages. Examples of the natural carbohydrates include monosaccharides such as glucose, disaccharides such as fructose, polysaccharides such as sucrose, conventional sugars such as dextrin and cyclodextrin, and sugar alcohols such as xylitol, sorbitol, and erythritol. Examples of the flavorings include natural flavorings (thaumatin, stevia extracts, such as rebaudioside A, glycyrrhizin, etc.) and synthetic flavorings (saccharin, aspartame, etc.). In addition, the food composition of the present invention may contain various nutrients, vitamins, minerals (electrolytes), flavorings such as synthetic flavorings and natural flavorings, colorants, pectic acid and its salt, alginic acid and its salt, organic acids, protective colloidal thickeners, pH adjusting agents, stabilizers, preservatives, glycerin, alcohol, carbonizing agents that are used in carbonated beverages, etc. Such components may be used individually or in combination. Although the content of these additives is generally selected in the range of 0.1 to about 100 parts by weight based on 100 parts by weight of the food composition of the present invention, without being limited thereto.

In one embodiment of the present invention, the “topical composition” refers to a composition that is used in various ways to prevent or ameliorate the indications targeted by the present invention, and for the purpose of the present invention, the topical composition of the present invention is for preventing or improving wet age-related macular degeneration. The topical composition containing the effective ingredient of the present invention includes a cosmetic composition applied to the external tissue of the eye, and a therapeutic preparation. When the topical composition is used as a therapeutic preparation, the most common form may be an ointment or artificial tears. The cosmetic composition is a composition for improving or alleviating wet age-related macular degeneration and other symptoms caused therefrom. The external preparation composition containing the effective ingredient of the present invention can be manufactured in the form of an ointment, artificial tears, toner, nutritional lotion, nutritional essence, massage cream, cosmetic bath additive, body lotion, body milk, bath oil, baby oil, baby powder, shower gel, shower cream, sunscreen lotion, sunscreen cream, suntan cream, skin lotion, skin cream, UV protection cosmetics, cleansing milk, depilatory agent {cosmetic}, face and body lotion, face and body cream, skin whitening cream, hand lotion, hair lotion, cosmetic cream, jasmine oil, bath soap, liquid soap, cosmetic soap, shampoo, hand cleanser (hand cleaner), medicated soap {non-medical}, cream soap, facial wash, hair rinse, cosmetic soap, tooth whitening gel, toothpaste, etc. To this end, the composition of the present invention can further contain an appropriate carrier, excipient or diluent commonly used in the manufacture of external preparation compositions. Carriers, excipients or diluents that may be further added to the external preparation composition of the present invention include, but are not limited to, purified water, oils, waxes, fatty acids, fatty alcohols, fatty acid esters, surfactants, humectants, thickeners, antioxidants, viscosity stabilizers, chelating agents, buffers, low-grade alcohols, etc. In addition, whitening agents, moisturizers, vitamins, UV blockers, perfumes, dyes, antibiotics, antibacterial agents, and antifungal agents may be included as needed. Examples of the oils that may be used include hydrogenated vegetable oils, castor oil, cottonseed oil, olive oil, palm kernel oil, jojoba oil, and avocado oil, and examples of the waxes that may be used include beeswax, ozokerite, carnauba wax, candelilla wax, montan wax, ceresin, liquid paraffin, and lanolin. As the fatty acid, stearic acid, linoleic acid, linolenic acid, and oleic acid can be used, and the fatty alcohols include cetyl alcohol, octyldodecanol, oleyl alcohol, panthenol, lanolin alcohol, stearyl alcohol, or hexadecanol can be used; and as the fatty acid ester, isopropyl myristate, isopropyl palmitate, or butyl stearate can be used. As the surfactant, cationic surfactants, anionic surfactants, and nonionic surfactants known in the art can be used, and surfactants derived from natural products are preferable if possible. In addition, hygroscopic agents, thickeners, antioxidants, etc. widely known in the field of external preparations can be included, and the types and amounts of these are as known in the art.

In one embodiment of the present invention, there is provided a pharmaceutical composition for preventing or treating wet age-related macular degeneration comprising nicotinamide, or derivatives thereof, as an active ingredient. In the pharmaceutical composition, the nicotinamide is a compound of Formula 1, and the derivatives of nicotinamide is a compound of Formula 2, or Formula 3. In the pharmaceutical composition, the wet age-related macular degeneration is accompanied by angiogenesis or fibrous scar formation.

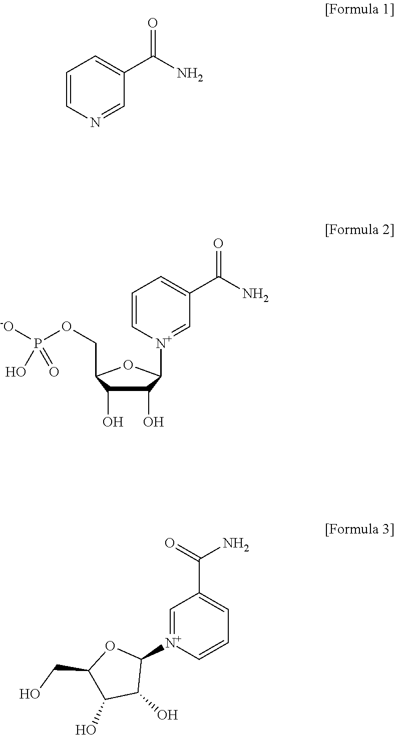

In another embodiment of the present invention, there is provided a food composition for preventing or ameliorating wet age-related macular degeneration comprising nicotinamide, or derivatives thereof, as an active ingredient. In the food composition, the nicotinamide is a compound of Formula 1, and the derivatives of nicotinamide is a compound of Formula 2, or Formula 3. In the food composition, the wet age-related macular degeneration is accompanied by angiogenesis or fibrous scar formation. And the food composition can be manufactured in the form of a beverage, gum, tea, vitamin complex, powder, granule, tablet, capsule, jelly, gel, confectionery, rice cake, or bread.

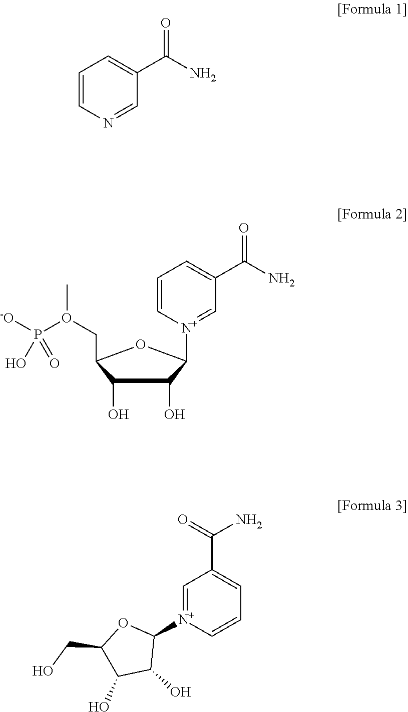

In a further embodiment of the present invention, there is provided a topical composition for preventing or ameliorating wet age-related macular degeneration comprising nicotinamide, or derivatives thereof, as an active ingredient. In the topical composition, the nicotinamide is a compound of Formula 1, and the derivatives of nicotinamide is a compound of Formula 2, or Formula 3. In the topical composition, the wet age-related macular degeneration is accompanied by angiogenesis or fibrous scar formation. And the topical composition can be manufactured in the form of an ointment or artificial tear.

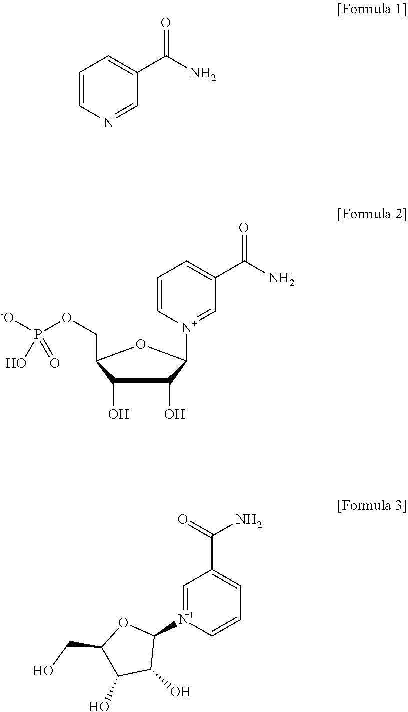

In another further embodiment of the present invention, there is provided a method for preventing or treating wet age-related macular degeneration, comprising a step of administering to a subject in need thereof a pharmaceutically effective amount of nicotinamide, or derivatives thereof. In the method, the nicotinamide is a compound of Formula 1, and the derivatives of nicotinamide is a compound of Formula 2, or Formula 3. In the method, the wet age-related macular degeneration is accompanied by angiogenesis or fibrous scar formation.

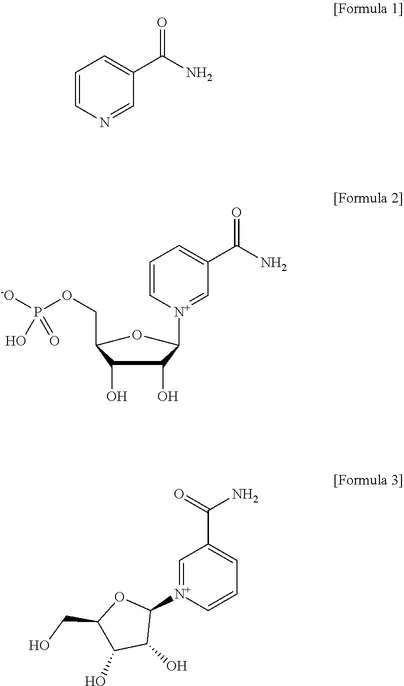

### Advantageous Effects

The pharmaceutical composition comprising nicotinamide, or derivatives thereof as an active ingredient according to the present invention has a remarkable effect on inhibiting angiogenesis and fibrous scar formation in wet age-related macular degeneration.

### BRIEF DESCRIPTION OF DRAWINGS

FIG. 1 shows that NMN and NR significantly reduce the size of neovessels, in accordance with one embodiment of the present invention.

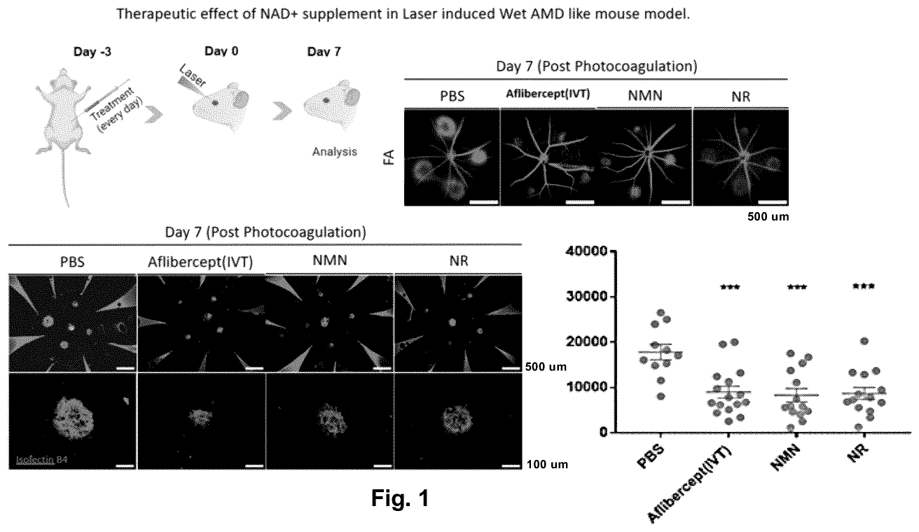

FIG. 2 shows results confirming that NMN inhibits the aging phenotype induced in a neovascular wet AMD model, in accordance with one embodiment of the present invention.

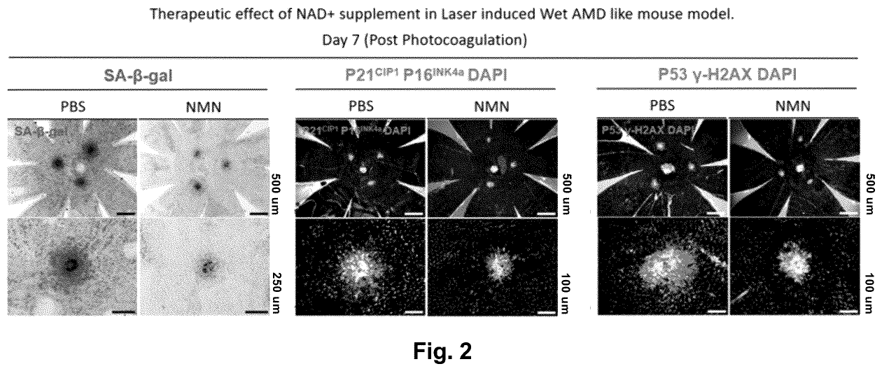

FIG. 3 shows results confirming that NMN and NR inhibit the production of subretinal fibrosis associated with neovascular macular degeneration, in accordance with one embodiment of the present invention.

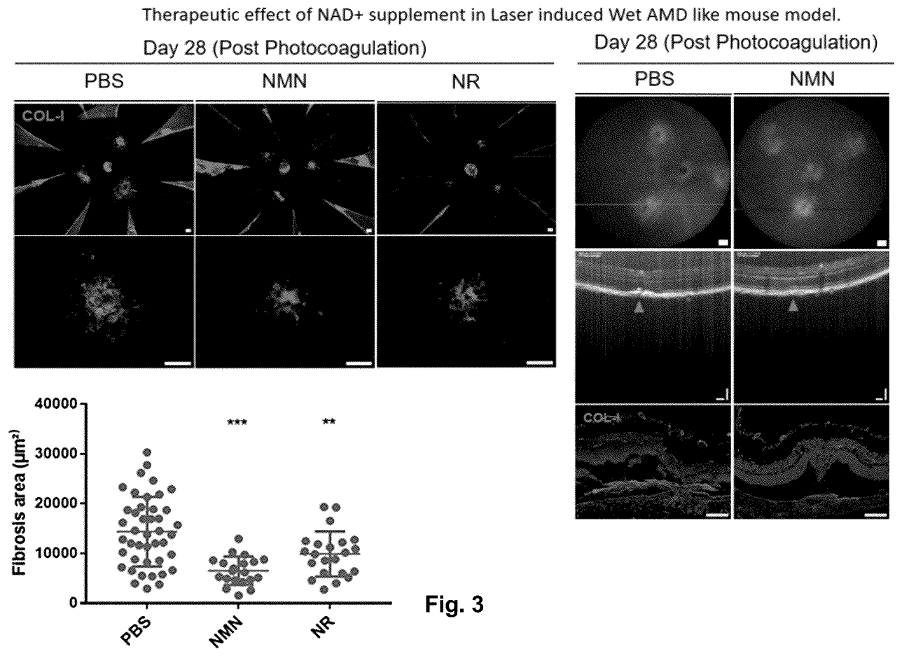

### MODE FOR INVENTION

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### Example

### Methods

### (1) Experimental Animals

In this study, 8-week-old (18-20 g) C57BL/6 J adult mice were used. The mice were maintained according to the guidelines for the use of animals in ophthalmic and vision research set forth by the Association for Research in Vision and Ophthalmology (ARVO). All animal experiments were approved by the Institutional Animal Care and Use Committee (IACUC) of Yonsei University College of Medicine (IACUC number: 2022-0279). C57BL/6 J mice were maintained on a 12:12 hour light/dark cycle.

### (2) Modeling Choroidal Neovascularization (CNV) and Fibrosis

Mice were anesthetized using a 1:1 mixture of tiletamine and zolazepam (15 mg per kg body weight) along with Xylazine hydrochloride (10 mg per kg body weight). The pupils were dilated using Phenylephrine (0.5%) and tropicamide (0.5%). During the anesthesia and pupil dilation, a heating pad was used to maintain the body temperature at 37° C.

After confirming pupil dilation, the mice were positioned on the support of the laser device (GYC-1000 Specifications, 532 nm). A drop of 2% Hypromellose solution was placed on the corneal surface, and a glass cover slip was positioned to contact the cornea for visualization of the fundus. Additionally, the height and distance of the support were adjusted to ensure the laser was accurately positioned on the retina.

The settings for the laser used in the experiment were as follows: output: 150 mW/spot size: 50 μm, time: 50 ms. After positioning the optic nerve in the center, the laser direction was indicated using a clock face, applying a total of 4 laser shocks at the 3 o'clock, 6 o'clock, 9 o'clock, and 12 o'clock positions, once each. The successful performance of the laser shock was confirmed by the generation of bubbles in the retina, with no hemorrhage observed in the retina.

To compare the choroidal neovascularization (CNV) induced by the laser shock, mice were sacrificed 7 days after the laser shock, and to evaluate the fibrosis induced by the laser shock in the sub-retinal area, mice were sacrificed 4 weeks after the laser shock.

### (3) Administration of NMN or NR

In this study, Nicotinamide mononucleotide (NMN) purchased from ORIENTAL YEAST CO and Nicotinamide riboside (NR) purchased from ChromaDex were used. Starting three days before the creation of the laser-induced mouse model, NMN and NR were administered intraperitoneally daily at a concentration of 500 mg per kg of body weight.

### (4) Immunofluorescent Staining

Mice were sacrificed using carbon dioxide, and the extracted eyeballs were fixed in 1% paraformaldehyde for 1 hour. After fixation, they were sequentially treated with 10%, 20%, and 30% sucrose solutions for 1 hour each. For sectioning, the eyeballs were positioned so that the connection between the optic nerve and the center of the cornea was horizontally aligned using OCT compound, and rapid freezing was performed using liquid nitrogen vapor. The eyeballs were stored at −70° C.

The eyeballs were sectioned to a thickness of 7 μM, and thereafter, staining procedures were performed using primary and secondary antibodies. After staining, a drop of mounting media was applied to the tissue on the slide, and a cover glass was placed over it.

For flat mounting, the muscle, cornea, lens, and neural retina were removed. Then, the retinal pigment epithelium/choroid/sclera complex was radially incised eight times to create a petal-like structure. It was placed in a 1 ml microtube and stained with primary and secondary antibodies.

The primary antibodies used in this study are as follows: Isolectin GS-IB4, Alexa Fluor™ 488 conjugate (121411); Collagen I antibody (ab34710); p16INK4a antibody (ab108349); gamma H2A.X antibody (ab2893); p53 antibody (sc-126); p21CIP1 antibody (sc-6246).

After the staining process was completed, the flat-mounted tissue was spread on the slide, a drop of mounting media was applied, and a glass cover was placed over it. Fluorescent images were taken using a fluorescence microscope (Olympus; BX43).

### (5) Histological Analysis

The size of neovascularization and fibrotic areas was measured using ImageJ software. Neovascular or fibrotic areas were selected, and the background was removed. Then, a threshold was applied to measure the size, followed by quantification of pixels.

### (6) SA-β-GAL Analysis

Mouse eyes were enucleated, and the anterior segments were removed to isolate the retinal pigment epithelium/choroid/sclera complex. The complex was rinsed with cold PBS and then fixed at room temperature for 20 minutes using the fixative solution provided in the SA-β-galactosidase (SA-β-GAL) staining kit. Staining was performed using the staining solution mixture provided according to the manufacturer's instructions (BioVision; #K320). After overnight staining at 37° C., the samples were incubated at 55° C. for 45 minutes with 10% H₂O₂ to remove the pigment. Following rinsing with PBS, the samples were flattened using forceps and a surgical knife. Images were captured using a fluorescent microscope (Olympus; BX43).

### (7) Statistics

All group results are presented as mean±standard error of the mean (S.E.M.).

Comparisons between groups were performed using a two-tailed Student's t-test or one-way analysis of variance (ANOVA), with Bonferroni post-hoc tests for multiple group comparisons. Statistical significance was indicated in figures and figure legends with asterisks: *(P<0.033), **(P<0.002), and ***(P<0.001). Statistical analyses were conducted using GraphPad Prism 7.0 software.

### Results

### (1) NMN and NR Significantly Reduce the Size of Neovascularization.

In this study, we used a choroidal neovascularization (CNV) mouse model of age-related macular degeneration (wet AMD) induced by laser. From 3 days before the laser intervention to 7 days after, NMN and NR were administered intraperitoneally daily at a dose of 500 mg per kg of body weight for a total of 10 days. PBS was used as a negative control group, while intravitreal Aflibercept injection was used as a positive control group. Seven days after the laser treatment, Fundus Fluorescein Angiography (FFA) analysis was performed to compare the size of neovascularization, and immunofluorescence staining using Isolectin B4 antibody was also conducted. In the NMN and NR groups, the size of neovascularization was significantly reduced compared to the PBS control group (NMN vs PBS, p<0.001; NR vs PBS, p<0.001). The Aflibercept group used as a positive control showed a significant reduction in neovascular size compared to the PBS group, but there was no significant difference compared to the NMN and NR groups (Aflibercept vs PBS, p<0.001; Aflibercept vs NMN, p>0.99; Aflibercept vs NR, p>0.99).

### (2) NMN Inhibits Age-Related Phenotypes Induced in a Neovascular Wet AMD Model.

SA-β-gal staining analysis, an early method for identifying senescent cells in tissues, was used. Additionally, the expression of senescence markers such as p16INK4a, p21CIP1, γ-H2AX, and p53 is increased in senescent cells. We examined whether NMN, which inhibits neovascularization, also inhibits aging mechanisms. PBS was used as a negative control group, and on the seventh day post-laser induction, SA-β-gal staining analysis and immunofluorescence staining were performed. As a result, in the NMN-injected mouse group, SA-β-gal staining of the retinal pigment epithelium/choroid/sclera complex was reduced compared to the PBS group. Additionally, the NMN-injected mouse group showed decreased expression of p16INK4a, p21CIP1, γ-H2AX, and p53 proteins compared to the negative control group injected with PBS.

(3) NMN and NR Inhibit the Formation of Subretinal Fibrosis Associated with Neovascular Age-Related Macular Degeneration.

In a laser-induced neovascular wet macular degeneration model, the neovascular area resulting from the shock of the laser can lead to subsequent subretinal fibrosis. This can occur or persist even after the current treatment for neovascular age-related macular degeneration, namely Anti-VEGF therapy, and may be a cause of limited visual improvement or vision loss after treatment. To confirm whether NMN and NR inhibit fibrosis, NMN and NR were injected intraperitoneally daily at a dose of 500 mg per kg of body weight from three days prior to the laser shock until the 28th day post-shock. PBS was used as a negative control. On the 28th day post-laser shock, immunofluorescence staining with Collagen type I antibodies, which mark fibrosis, and optical coherence tomography (OCT) to assess the progression of fibrosis were performed. The results showed that in the mouse groups injected with NMN and NR, the expression of Collagen type 1 was significantly reduced compared to the PBS negative control group (NMN vs. PBS p<0.001, NR vs. PBS p<0.002). Additionally, OCT and immunofluorescence staining on sections confirmed that fibrosis was reduced in the NMN-treated mouse group compared to the negative control group.

From the results of the above example, it can be concluded that nicotinamide derivatives, such as nicotinamide mononucleotide (NMN) or nicotinamide riboside (NR), have significant effects in the prevention or treatment of wet age-related macular degeneration accompanied by neovascularization and fibrosis.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1. A pharmaceutical or food composition for preventing or treating wet age-related macular degeneration comprising nicotinamide, or a derivative thereof, as an active ingredient.

2. The pharmaceutical or food composition according to claim 1, wherein the nicotinamide is a compound of Formula 1:

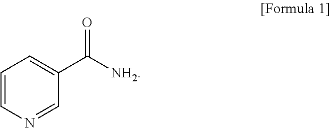

3. The pharmaceutical or food composition according to claim 1, wherein the derivative of nicotinamide is a compound of Formula 2, or Formula 3

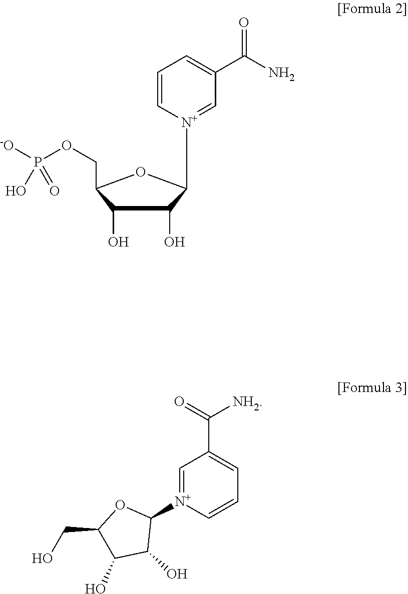

4. The pharmaceutical or food composition according to claim 1, wherein the wet age-related macular degeneration is accompanied by angiogenesis.

5. The pharmaceutical or food composition according to claim 1, wherein the wet age-related macular degeneration is accompanied by fibrous scar formation.

6-10. (canceled)

11. The pharmaceutical or food composition according to claim 1, wherein the pharmaceutical or food composition is a food composition manufactured in the form of a beverage, gum, tea, vitamin complex, powder, granule, tablet, capsule, jelly, gel, confectionery, rice cake, or bread.

12. A topical composition for preventing or ameliorating wet age-related macular degeneration comprising nicotinamide, or derivatives thereof, as an active ingredient.

13. The topical composition according to claim 12, wherein the nicotinamide is a compound of Formula 1:

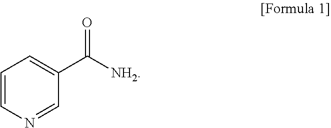

14. The topical composition according to claim 12, wherein the derivatives of nicotinamide is a compound of Formula 2, or Formula 3:

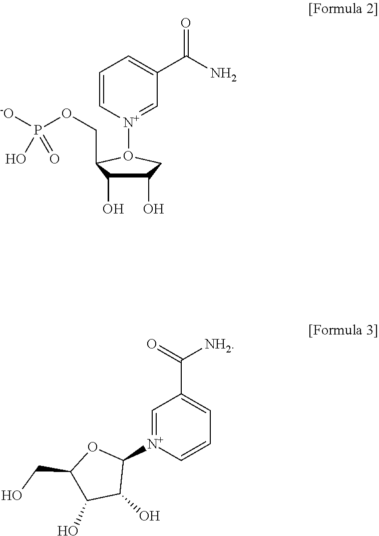

15. The topical composition according to claim 12, wherein the wet age-related macular degeneration is accompanied by angiogenesis.

16. The topical composition according to claim 12, wherein the wet age-related macular degeneration is accompanied by fibrous scar formation.

17. The topical composition according to claim 12, wherein the topical composition is manufactured in the form of an ointment or artificial tear.

18. A method for preventing or treating wet age-related macular degeneration, comprising a step of administering to a subject in need thereof a pharmaceutically effective amount of nicotinamide, or a derivative thereof.

19. The method according to claim 18, wherein the nicotinamide is a compound of Formula 1:

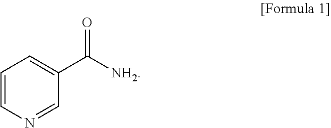

20. The method according to claim 18, wherein the derivative of nicotinamide is a compound of Formula 2, or Formula 3:

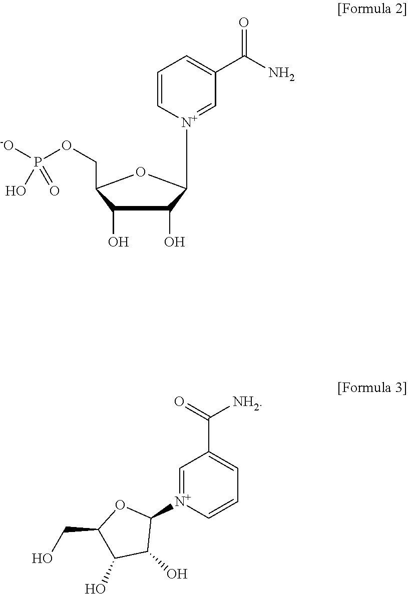

21. The method according to claim 18, wherein the wet age-related macular degeneration is accompanied by angiogenesis.

22. The method according to claim 18, wherein the wet age-related macular degeneration is accompanied by fibrous scar formation.

23. The method according to claim 18, wherein the administration is performed by administering a pharmaceutical composition or a food composition comprising the nicotinamide or derivative thereof.

24. The method according to claim 18, wherein the administration is performed by administering a topical composition comprising the nicotinamide or derivative thereof.

25. The pharmaceutical or food composition according to claim 1, wherein the wherein the pharmaceutical or food composition is a pharmaceutical composition.
